(19) United States
(12) Patent Application Publication
(10) Pub. No.: US 2021/0325369 A1
(43) Pub. Date: Oct. 21, 2021

(54) PREDICTIVE BIOMARKERS FOR AN IMMUNE RESPONSE

(71) Applicant: HUMAN VACCINES PROJECT, New York, NY (US)

(72) Inventors: Wayne C. Koff, New York, NY (US); Richard H. Scheuermann, La Jolla, CA (US); Tobias Kollmann, Vancouver (CA); Casey P. Shannon, Vancouver (CA); Scott J. Tebbutt, Vancouver (CA)

(21) Appl. No.: 17/258,871

(22) PCT Filed: Jul. 29, 2019
(86) PCT No.: PCT/US19/43838

§ 371 Date: Jan. 8, 2021

Related U.S. Application Data
(60) Provisional application No. 62/711,048, filed on Jul. 27, 2018.
(60) Provisional application No. 62/752,477, filed on Oct. 30, 2018.

(51) Int. Cl. G01N 33/50; A61K 45/06; A61K 39/39; A61K 39/29; A61P 31/20; C12Q 1/6876; A61K 39/015; A61P 33/06; A61K 39/245; A61P 31/22

(52) U.S. Cl. CPC G01N 33/5023; A61K 45/06; A61K 39/39; A61K 39/292; A61P 31/20; C12Q 1/6876; A61K 2039/555; A61P 33/06; A61K 39/245; A61P 31/22; G01N 2800/52; C12Q 2600/106; C12Q 2600/154; A61K 39/015

(57) ABSTRACT

Biomarkers and uses thereof, as well as methods for using same for identifying vaccine recipients who will respond to a single dose of vaccine. In addition, an integration model for identifying biomarkers is also provided, such that the biomarkers form a network of signatures associated with a vaccine responder.

### CROSS-REFERENCE TO RELATED APPLICATION

This application is a national phase application of PCT Application No. PCT/US2019/043838, internationally filed Jul. 29, 2019, which claims priority to Provisional Patent Application No. 62/711,048, filed Jul. 27, 2018, and Provisional Patent Application No. 62/752,477, filed Oct. 30, 2018, all of which are herein incorporated by reference in their entirety.

### FIELD OF THE INVENTION

The Present invention provides a network of biomarkers that can predict an immune response to a vaccination in a subject. The biomarkers are useful as indicators of when a subject's immune system will respond to a vaccination, and as a setpoint for pre-vaccination modulation, where the setpoint is the target for immune modulation prior to a vaccination.

### BACKGROUND OF THE INVENTION

The influenza pandemic of 1918 resulted in the infection of over a third of the world's population, and resulted in the death of between 50 and 100 million people. Today, influenza continues to loom as a significant global threat, resulting in the annual, seasonal death of between 250,000 and 500,000 people worldwide, as well as millions of people suffering from the symptoms of the infection.

Vaccines preventing infections or disease, like the influenza pandemic, are amongst the most effective life-saving medical interventions in history. S. Plotkin, History of vaccination. Proc Natl Acad Sci USA 111, 12283-12287 (2014). In the past, vaccine design was largely empiric. However, this approach has thus far largely failed to tackle complex infections such as human immunodeficiency virus (HIV), tuberculosis (TB), and malaria, as well as cancer and other non-communicable diseases. The failure has been attributed to the lack of insight into the underlying mechanisms of how vaccines induce protection. W. C. Koff et al., Accelerating next-generation vaccine development for global disease prevention. Science 340, 1232910 (2013). Recent systems immunology advances including highly multiplexed immune profiling and data-driven computational modeling have raised the prospect of identifying some of these mechanisms. However, given the extensive population heterogeneity, being able to identify these mechanisms and being able to predict who would respond to a vaccine is a daunting task. Take the example of influenza again, influenza can result in mild to severe respiratory distress, as well as numerous serious complications (pneumonia and secondary bacterial infections, for example). The Influenza virus is combatted using immunization by yearly influenza vaccines, a process started in the 1930s and 40s. Current vaccines are based on immunizing against three different types of virus, influenza A (H1N1), influenza A (H3N2), and influenza B viruses. Within each of these types of virus, there are many different strains, which are constantly in flux (antigenic drift), resulting in a significant number of potentially different antigens (antibody-binding or recognition sites) on the coat of any one virus. How any one person's immune system will respond to this virus requires knowledge on how that person's immune system is positioned at the time of vaccination. Much like the game of chess, an understanding of how the pieces move and a strategy for why and when to move a piece is needed to play the game effectively, the same understanding is required with respect to the immune system.

Influenza vaccines are based on a limited understanding of the years influenza virus strain, and on how the average person's immune system is thought to work. In most cases, the vaccine for the upcoming influenza season is developed on strain information available in the spring, so as to provide enough time to produce and distribute the vaccination materials. In other cases, e.g., tropic and sub-tropic areas, the potential for influenza outbreak is year round, further limiting the ability of vaccine design to match active viral strains. As noted above, the influenza virus is constantly going through antigenic drift, resulting in only some level of match between the virus and vaccine, regardless of the location of the outbreak. Where a match is imperfect, the vaccine may only have modest effects on immunizing the recipient, and in some cases, little or no effect on immunizing the recipient. In addition, regardless of the vaccine-virus match, some vaccine recipients will not respond to the vaccine, these recipients having a population of B and T cells that inherently do not respond to the particular vaccine/virus. It is believed that the human immunome has up to 10-20 million clonotypes of B cell and T cell receptors.

In this light, the immune system of vaccine recipients undergoes changes to a number of underlying genes in their innate and adaptive immune cells, thereby preparing the recipient for subsequent infection with the virus. These same factors are involved in almost every immunization, whether it is for influenza, pertussis, polio, HIV, hepatitis B, or any other infectious agents. As such, a better understanding of the immune system and biomarkers that identify a vaccine recipient as a responder to a particular vaccine, are of paramount importance to the field of infectious diseases. In the example of the influenza vaccine, verifying whether a subject will respond to a vaccine prior to its administration allows health care professionals to target individuals in need of additional follow-up or, alternatively, not in need of any additional care. In addition, identification of the network of biomarkers involved in the immune response to viral infections, allows for a more efficient and robust vaccine design and development program. These findings can be extended to cancer treatment as well as to other non-communicable diseases.

In this light, there is a need in the art for a better understanding of what immune system changes, including epigenetic changes, cell immunity changes, and genetic changes are being modulated by vaccines in order to identify vaccine responders and to find a more efficient and universal vaccine development strategy. In addition, identifying an immune setpoint for a positive outcome to a vaccination allows for modulation of a subject's immune system network to that setpoint, prior to vaccination. Identification of how to pre-set a subject's setpoint could lead to a dramatic increase in vaccination efficiency.

Against this backdrop the present disclosure is provided.

### SUMMARY OF THE INVENTION

Embodiments herein provide universal biomarkers for identifying a mammal, and typically a human, that will respond to a vaccine. In embodiments herein, a mammal that will be receiving a vaccination is referred to as a subject or recipient. These biomarkers provide predictive measures for determining whether a subject will respond to a vaccine, and typically, will respond with two or fewer doses, and more typically, one dose of the vaccine. These biomarkers can also be used to develop universal testing procedures for predicting whether a vaccine is eliciting a proper response from a vaccine in a population of subjects. In addition, the biomarkers can also represent a setpoint for maximizing efficiency of a vaccination in a subject, a setpoint that can be obtained through the use of immune modulators prior to a vaccination. Aspects of these embodiments can include one, two or more, three or more, four our more, and the like, biomarkers that can represent a network of indicators for both the prediction on whether a subject will respond to a vaccine, or act as a setpoint for a subject, obtainable prior to vaccination through the use of immune modulators.

In this light, embodiments herein provide biomarkers for identifying a subject who will respond to a vaccine, i.e., is a subject's immune system receptive to response to a particular vaccine or vaccine antigen. In some aspects, the biomarkers are identified within a sample obtained from the subject, where the sample may be peripheral blood, saliva, urine, lymph, lymph nodes, spleen, bone marrow, and the like. Biomarkers are identified using reagents specific for each of the below identified biomarkers, including antibodies, primers, probes, etc.

In a first aspect, the biomarker is GFI1, and an increase in GFI1 in a blood sample from a subject, over a control or median amount of GFI1, or appropriate for a vaccine responder, is indicative of a subject that will respond to a vaccine; in another aspect, the biomarker is GFI2, and an increase in GFI2 in a blood sample from a subject, over a control or median amount of GFI2, or appropriate for a vaccine responder, is indicative of a subject that will respond to a vaccine; in another aspect, the biomarker is GF15 (CD82), and an increase in GF15 in a blood sample from a subject, over a control or median amount of GF15, or appropriate for a vaccine responder, is indicative of a subject that will respond to a vaccine; in still another aspect, the biomarker is CD8a, and an increase in CD8a in a blood sample from a subject, over a control or median amount of CD8a, or appropriate for a vaccine responder, is indicative of a subject that will respond to a vaccine; in yet another aspect, the biomarker is CD8b, and an increase in CD8b in a blood sample from a subject, over a control or median amount of CD8b, or appropriate for a vaccine responder, is indicative of a subject that will respond to a vaccine; and in still yet another aspect, the biomarker is LGALS3, and a decrease in LGALS3, over a control or median amount of LGALS3, or appropriate for a vaccine responder, is indicative of a subject that will respond to a vaccine.

In addition, any combination of two or more of the above biomarkers, three or more of the above biomarkers, four or more of the above biomarkers, and so on, can be used to identify a subject that will respond to a vaccine. In such instances, the combination of biomarkers forms a network of indicators that can be used to predict the outcome of a vaccination for a subject. For example, an increase in both biomarkers GFI1 and GF15 in the blood of a subject, over a control or median amount of GFI1 and GF15, or having a GFI1 and GF15 signature appropriate for a vaccine responder, is indicative of a subject that will respond to a single dose of vaccine. As noted, the network of indicators can be three or more biomarkers used to identify a subject that will respond to a vaccine. For example, an increase in biomarkers GFI1 and GF15, and a decrease in the LGALS3 biomarker, in the blood of a subject, over a control or median amount of each, or having a signature appropriate for a vaccine responder, is indicative of a subject that will respond to a single dose of vaccine. In some cases, four or more biomarkers, five or more biomarkers, six or more biomarkers, seven or more biomarkers, and the like, can be used to identify a subject that will respond to a vaccine. The biomarkers form a network of indicators to be used in predicting whether an immune system is receptive to being immunized by a vaccine.

Embodiments herein also provide biomarkers associated with blood myeloid dendritic cells (mDCs) that can be used to identify a subject who will respond to a vaccination. In some aspects, the mDCs are identified within a sample of peripheral blood of the subject, and then further tested for mDC specific biomarkers. In one aspect, the biomarker in mDCs is a decrease in CDKN1 in a subject, over a control or median amount of CDKN1, or an amount appropriate for a vaccine responder, is indicative of a subject that will respond to a vaccine; and in yet one more aspect, the biomarker in mDCs is an increase in NDRG2 in a subject, over a control or median amount of NDRG2, or having an amount appropriate for a vaccine responder, is indicative of a subject that will respond to a vaccine. In some cases, a decrease in CDKN1 and increase in NDRG2 in a subject, over a control or median amount of each, both in line with other vaccine responders, is indicative of a subject that will respond to a vaccine. In still another embodiment, it is a ratio of NDRG2/CDKN1 in mDCs that is indicative of a responder to a vaccine. Also, an increase in FCER1A in a subject is indicative of a responder to a vaccine, and a decrease to FCGR3A in a subject is indicative of a responder to a vaccine, as compared to a control or median amount (and in line with other vaccine responders). In addition, the ratio of different subsets of mDC cells, DC2 and DC4, found in whole blood, also provide a predictive measure for identifying a subject who will respond to a vaccine. In some embodiments, the network of biomarkers is a combination of the four genes, CDKN1, NDRG2, FCGR3A and FCER1A and the relative numbers of DC2 and DC4 subpopulations of mDC cells in the subject.

Embodiments herein also provide epigenetic indicators that a subject will respond to a vaccination. As above, sample testing can be performed on peripheral blood of a subject. In one aspect, hypermethylation of the RPS14 ribosomal gene in the blood of a subject, as compared to a control or median amount of hypermethylation in RSP14 ribosomal gene, is indicative that a subject will respond to a vaccine. Also as above, the amount of hypermethylation in the RPS14 ribosomal gene is similar to hypermethylation found in other known vaccine responders (as compared to non-responders).

Embodiments herein also provide indicators based on the level of stimulated CD4 T cells to predict whether a subject will respond to vaccination. In one aspect, use of the Activation Inducted Markers Assay (AIM Assay) predicts the number of CD25+OX40+ T cells in a subject, where an increase in CD25+OX40+ cells over a median number of cells in like subjects, is indicative of a subject who will respond to a vaccine, and in line with other know responders to vaccines.

Embodiments herein also provide a network of indicators or biomarkers, identified using an integration model, for predicting whether a subject will respond to a vaccination. The network can include biomarkers from gene expression, biomarkers based on epigenetics, biomarkers based on numbers of different types of T cells or mDC cells, and markers associated with or on specific types of cells, for example, biomarkers specific to mDC cells. The network of biomarkers are integrated and a signature for vaccine response identified. In some aspects, the biomarkers are integrated to provide the signature of a responder using a Latent cOmponents integrative method (see below).

Embodiments herein also provide methods for predicting a response of a vaccine in a subject in need of a vaccination. A subject that will respond to a vaccine is one who will respond after two or fewer vaccinations, and more typically after a single vaccination. Methods include use of one or more of the biomarkers, e.g., mRNA transcripts, epigenetic markers, number of activated T cells, or AIM assay markers to identify a subject who will respond to a vaccination after a single dose. Response to a vaccine includes the subject having sufficient immunity to not require additional vaccine doses, at least over the course of the initial year after immunization. A comparison of the one or more biomarkers, including epigenetic markers, activated T cells, or AIM assay markers identified in the subject, as compared to a control or median amount of each, determines whether a subject will respond to the vaccine. Once a prediction is made, the subject can be immunized with the vaccine as determined by whether the subject is a responder or non-responder to the vaccine.

In one aspect, a method for identifying a vaccine responder comprises: isolating a peripheral blood or other appropriate sample from a subject; contacting the peripheral blood with reagents specific for one or more of the biomarkers described above to assess the biomarker; compare the assessed biomarker in the sample to the same biomarker in a control sample for the biomarker; and determining whether the comparison of the sample biomarker to the control biomarker is a positive/negative indicator of a vaccine responder. In some cases, a control is not used, but rather a known comparison to other vaccine responders is used to indicate whether the subject is a vaccine responder. In some methods, one or more, two or more, three or more, four or more, and the like, biomarkers can be assessed as indicators of whether a subject is a responder.

In an alternative embodiment, a method for identifying a network of biomarkers in a vaccine responder comprises: isolating one or more samples from a series of subjects to be tested prior to vaccination, and isolating one or more samples from the same subjects after vaccination; testing the one or more samples from both prior to and after vaccination for a series of molecular and cellular biomarker identification tests; identifying which subjects responded to the vaccination through antibody titer; using an integrative data model to accurately predict which biomarkers associate with a vaccine response; and repeating the method on a new subject to continue to expand the biomarker network known to associate with vaccine response.

In addition, embodiments herein provide kits for identifying a responder to a vaccine. Kits include reagents for detecting and assessing at least one of the biomarkers described herein. Kits may include antibodies to CD8a, CD8b, NDRG2, for example, or reagents necessary to identify the methylation status of a target biomarker. In one aspect, kits may also include an appropriate vaccine tied to the kits use, or sample collection devices, e.g., blood collection syringes, swabs, etc. Further, kits may include instructions on the control levels for biomarkers. In some assays, the kits include solid substrate biomarker reagent array plates for running a battery of biomarker evaluations on a sample.

Embodiments herein also include methods for altering a subject's biomarker setpoint to alter or improve the outcome of a vaccination. In this embodiment, one or more samples are recovered from a subject in need of a particular vaccination; the samples are recovered prior to the vaccination, and a series of two or more, three or more, four or more, and the like, assays are performed on the samples to identify a baseline set of biomarkers, as described herein the subject's baseline network of indicators are then compared to the same indicators for a vaccine responder, and a determination on whether the subject requires immune modulators to conform his or her biomarker setpoint network to a responders network; where required, the subject is administered one or more immune modulators to conform the subject's biomarker network to a responder's biomarker network. In some aspects, a second sample is taken from the subject after a predetermined amount of time to track the modifications of the subject's network of biomarkers, and where appropriate, challenged with a second administration of immune modulators. The vaccine is administered once the subject's biomarker network is comparable to a responders biomarker network.

Various features, aspects and advantages of the subject matter of embodiments herein will become more apparent from the following detailed description and claims, along with the accompanying figures.

### BRIEF DESCRIPTION OF THE DRAWINGS

FIG. 1 shows s series of regression plots on data from flow cytometry, DNA methylation, mRNA expression, microbiome, WBC lipidomics, and PLS lipidomics. The data indicates that the DIABLO integration model closely predicted which biomarkers would be predictive of actual antibody response. Biomarkers were enriched for genes involved in regulation of cytokine production and maturation of B-cells to plasma cells.

FIG. 2 shows an illustration on how modulating an immune baseline can modulate a vaccine response.

### DETAILED DESCRIPTION OF THE INVENTION

All publications, patents and patent applications cited in this document are hereby incorporated by reference in their entirety. The application having Ser. 62/711,048, filed on Jul. 27, 2018, and entitled “Methods and Kits for Facilitating Vaccine-Based Immunization,” is incorporated by reference in its entirety as is the application having Ser. 62/752,477, filed on Oct. 30, 2018, and entitled “Methods, Compositions, and Kits for the Identification of Vaccine Responders.”

Reference will now be made in detail in representative embodiments illustrated herein. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Unless defined otherwise, all technical and scientific terms used herein have the same general meaning as commonly understood by one of ordinary skill in the art to which embodiments herein belong.

Understanding the details of how the immune system works is an evolving and continuous investigation. This is particularly true with respect to adaptive immunity, and how vaccines are used to provide protection against particular infections. The inventors of the embodiments described herein have identified universal markers or biomarkers that can be used to predict the response of a vaccine in a recipient, and further, can be used to set a subject's biomarker network prior to administration of a vaccine, so as to maximize the vaccine's effectiveness. Further, these same biomarkers can be used in vaccine design and testing, to determine if a particular antigen elicits the predictive response of the biomarkers described herein. Vaccines that positively effect the identified biomarkers herein, are more likely to elicit a response from a population, than vaccines that have little or no effect on the biomarkers described herein.

Biomarkers are identified in samples isolated from a subject, where samples may include a number of different biologic sources in the subject. Samples herein may be collected or isolated from peripheral blood (whole blood, serum, plasma, or cellular components), body fluids, lymph, urine, saliva or tissue, e.g., lymph node aspirate, bone marrow, spleen, etc. Note, for example, that a single sample of blood can be used to perform a number of different tests: transcriptomics, epigenetics, proteomics, flow cytometry, plasma proteomics, metabolomics, and the like. In typical embodiments herein, a subject is screened for baseline biomarker status at 12 to 14 days prior to a vaccination, at which time approximately 100 ml, or so, of blood is harvested for testing, as well as lymph node aspirates and other source material. Post vaccination testing on the subject is typically performed on 50 ml of blood taken at days 1, 3, 7, 14 and 28 post vaccine administration. Day 14, or thereabout, post vaccination is also where a lymph node aspirate would be harvested for post-vaccination marker identification. Where a second vaccination is performed on the same subject, the vaccine is administered at 28 days after the first vaccination. Note that the above days for obtaining a sample in relation to a vaccination can be modified and are provided as an illustrative guide to one such pattern.

Embodiments in accordance with the present invention are directed to identifying various biomarkers in vaccine recipients associated with a greater response to a vaccine, as compared to other recipients having a below average response to the same vaccine. A response as referred to herein, is considered effective where a vaccine recipient only requires two or less, and more typically, one vaccination, over the course of one year, to resist and/or prevent the viral infection. In some embodiments, a responder is a vaccine recipient that requires only one vaccination over the course of one year, and more typically one vaccination over two or more years, three or more years, four or more years, and up to a lifetime, to resist and/or prevent viral infection. A vaccine recipient that responds in such a way is termed a “responder.” Conversely, a non-responder is a recipient that requires three or more vaccinations over the course of a year and may require additional testing to identify when the non-responder is fully immunized against the virus of interest, for example.

In some embodiments, it is understood that testing and determination of a biomarker in control samples has been determined by finding the average level of the biomarker in all tested subjects. Responders are those subjects that have an increase or decrease of a particular biomarker away from that calculated average, prior to being vaccinated. In some cases, biomarkers were identified by comparing biomarker levels or numbers in subjects having high cell mediated and humoral immunity to the vaccine, as compared to subjects that showed little or no change in the same biomarker, and also showed no increase in cell mediated and humoral immunity to the vaccine. These values were then evaluated against the biomarkers at pre-vaccination, to identify the appropriate correlation, i.e., known biomarker levels for responders can be compared to a subject's same biomarker level to see if they are comparable. Finally, as described in greater detail below, biomarkers can be identified using an integration model that maximizes the covariance between all data at pre-immunization sampling and at post-vaccination sampling. This data is then aligned with whether a tested subject actually responded to a vaccine by testing the subject's antibody titer. The model can then identify biomarkers associated with antibody response and look for the ones with the greatest covariance pre and post vaccination.

Embodiments in accordance with the present invention also include that a responder to a vaccine is a subject that prior to receiving the vaccine has a statistically significant change in various biomarker patterns (also termed networks), as compared to controls, that predict immunity after two or fewer, and more typically, a single dose of vaccine. A dose as referred to herein, is a conventional amount, administration route and administration site for the vaccine at issue. Using any one of the above methodologies, the following biomarkers have been identified:

In one aspect, an increase of the transcriptional suppressor GFI1 is an indication that a subject will be a responder to a vaccine; in another aspect, an increase of the transcriptional suppressor GFI2 is an indication that a subject will be a responder to a vaccine; in yet another aspect, an increase of the transcriptional suppressor GF15 is an indication that a subject will be a responder to a vaccine; in another aspect, an increase of CD8a and/or CD8b is an indication that a subject will be a responder to a vaccine; and in yet another aspect, a decrease in LGALS3 is an indication that a subject will be a responder to a vaccine. In some embodiments, amounts of biomarkers are found in mDCs, for example, decreases in CDKN1 and/or FCGR3A and increases in NDRG2 and FCER1A in mDCs, are indicative that a subject will respond to a vaccine. In some cases, it is the ratio of NDRG2/CDKN1 that is used to identify a subject that will respond to a vaccine.

Embodiments in accordance with the present invention also include a response as effective where the vaccine recipient has a pre-vaccine epigenetic modification of certain genes, as compared to controls. For example, a vaccine recipient that has a pre-vaccine hypermethylated RPS14 is an indication that the subject will be a responder to a vaccine.

In another embodiment, a response is considered effective where the vaccine recipient has a statistically significant increase in aspects of his or her pre-vaccination cell mediated immunity (CMI). In one aspect, an increase in cell mediated immunity is one where the vaccine recipient has a statistically significant increase in the numbers of CD25+OX40+ T cells (as compared to the number of these cells prior to the same vaccination or to a baseline number or median number for non-vaccinated subjects). In yet another embodiment, a response can be considered effective where subsets of mDC cells are increased in pre-vaccination recipients, for example DC2 and DC4 cells.

These and other aspects of embodiments herein will now be discussed in greater detail, after a review of a number of helpful definitions:

### Definitions

The use of the word “a” or “an” when used in conjunction with the term “comprising” in the claims and/or the specification may mean “one,” but it also consistent with the meaning of “one or more,” and “one or more than one.”

The term “providing” refers to its ordinary meaning to indicate “to supply or furnish for use.”

The term “administering” refers to the process of injecting, infusing, ingesting, mucosal contact, and the like of a material, e.g., vaccine, adjuvant, immune modulator, etc. to a subject.

The terms “marker” and “biomarker” are used interchangeably throughout the disclosure. A “marker” herein is any biologic parameter positively or negatively correlated with an immune response or with a subject's immune setpoint.

The term “responder” herein refers to a subject that shows antibody serum levels associated with immunity to an antigen after a single, medically approved, dose of a vaccine. The antibody titer associated with immunity is typically greater than 3 mIU/ml, and more typically greater than 10 mIU/ml. A responder is also a subject that has been identified as having one or more, two or more, three or more, four or more, and the like, biomarkers identified as being receptive to a vaccine.

The term “subject” refers to an individual able to receive one or more vaccinations with a predetermined vaccine and is typically a mammal, and more typically a human.

The term “correlate” or “correlation” or equivalents thereof refers to an association between an objective parameter for an immunity biomarker and a predicted antibody serum level for a target antigen or vaccine.

The term “immune modulator” refers to any substance that can positively modify a biomarker, or set of biomarkers, in a subject to correlate to a known parameter, or set of parameters, found in a vaccine responder. In some cases, the immune modulator is poly IC, CpG oligonucleotides, TLR9 agonist (lefitolimod, tilsotolimod), TLR3 agonists, imiquimod (TLR7) and/or pidotimod (TLR2 agonist), which is used to stimulate PBMCs, in other cases, the immune modular is an adjuvant (without antigen), like aluminum salts or MF59.

The term “influenza” refers to any of the highly contagious, respiratory diseases caused by any of the three, or related, orthomyxoviruses, influenza A, influenza B or Influenza C.

The term “Hepatitis B” refers to a disease caused by the Hepatitis B Virus (HBV) of the genus Orthohepadnavirus, family Hepadnaviridae, where the disease is marked by fatigue, fever, vomiting, darkened urine, jaundice and joint pain, which is often accompanied by liver damage and failure.

The term “control” refers to a level or amount of a biomarker associated with an average person, responder or non-responder, across a population of subjects.

The term “DIABLO” refers to a computational model using continuously expanding molecular and cellular data to identify biomarkers predictive of a vaccine responder. The model is described in: Singh et al., Diablo: an integrative approach for identifying key molecular drivers from multi-omics assays, Bioinformatics, 2019, 1-8, which is incorporated by reference in its entirety for all purposes.

The term “CD8A” refers to a cell surface glycoprotein that can be found on cytotoxic T cells and is a known mediator of cell-cell interactions within the immune system. The CD8A antigen is a biomarker for a responder herein.

The term “CD8B” refers to a cell surface glycoprotein that can be found on cytotoxic T cells, and like CD8A, is known as a mediator of cell-cell interactions within the immune system. The CD8B antigen is a biomarker for a responder herein.

The term “LGALS3” or “galectin 3” refers to a carbohydrate binding protein known to facilitate the differentiation of monocytes into dendritic cells. The LGALS3 antigen is a biomarker for a non-responder herein.

The term “NDRG2” refers to a member of the alpha/beta hydrolase superfamily of genes. The protein encoded by the NDRG2 gene is a cytoplasmic protein and is a biomarker in mDC cells in responders.

The term “FCGR3A” (also known as CD16a) refers to a cell surface biomarker often found on monocytes or natural killer cells. The biomarker is typically lower in mDC cells of responders.

The term “CD3-e” refers to the T cell surface glycoprotein CD3 epsilon chain.

The term “CDKN1” or “CDKNla” refers to a cyclin dependent kinase 1a.

The term “GRIl” refers to the growth factor independent 1 transcriptional repressor with chromosomal location 1p22.1.

The term “GFI2”, also known as ARHGAPS, refers to the RhoGTPase activating protein 5 with chromosomal location 14q12.

The term “RPS14” refers to the ribosomal protein S14 with chromosomal location 5q33.1.

The term “FCER1A” refers to the Fc fragment of IgE and is considered a potent effector of hypersensitivity. The biomarker is typically higher in mDC cells of responders.

The term “kit” refers to one or more biomarker detection assays and instructions for their use. The instructions may include product inserts, instructions on how to use or perform an assay, and/or instructions on how to analyze an assay. Kits can also include reagents required to perform any of the assays in accordance with embodiments herein.

The term “about” refers to a value within 10% of the numerical value being used. Therefore, about 50% means in the range 45% to 55%.

### Biomarker Identification Using Integrated Data Sets

As noted above, biomarkers (as obtained from any one biologic assay, including CBC, FCM, DNA methylation, microbiome, mRNA transcripts, WBC lipidomics, and PI Lipidomics) have been identified using comparisons of the biomarker in a population of known responders against the same biomarker in a population of non-responders. Where the biomarker has a statistical difference between the two groups it is correlated with vaccine response, as opposed to non-markers that remain the same, or relatively the same, between the two groups. Alternatively, markers have been identified by having an increase or decrease as compared to an average or control amount or a marker. So, a marker would show an increase or decrease against the average, where a non-marker would typically be the same value as the average or control amount. Finally, in some embodiments, data integration using computational and statistical strategies have been used to identify biomarkers and biomarker networks indicative of a vaccine response. In one aspect, the computational and statistical approach is the Data Integration Analysis for Biomarker Discovery Using Latent cOmponents (or DIABLO) multi-omics integrative method. As described herein, data can be obtained from a number of different sources and assays including CBC, FCM, DNA methylation, the microbiome, mRNA transcripts, WBC lipidomics, Plasma Lipidomics, WBC proteomics, Plasmas Proteomics. For example, thousands of data points can be evaluated from a single subject at a time prior to a first vaccination and at a time, one day, several days, fourteen days, etc. after a first vaccination.
The integrative model then takes the data and identifies one or more, two or more, three or more, four or more, five or more, six or more, seven or more, eight of more, and the like (i.e., a network), of biomarkers that have superior biological relevance for predicting whether a subject will respond to a vaccine.
In one embodiment, the data set was obtained from 15 subjects, where samples were obtained at pre-vaccine, one day post vaccine after one dose of HBV vaccine, three days post vaccine, 7 days post vaccine and 14 days post vaccine. Test were performed on the eight data types discussed above. A significant number of biomarkers were integrated using the model as is shown in Table 1 below.

TABLE 1
Data Available for Integration

| Data Type | # of Biomarkers Evaluated By Model |
|---|---|
| CBC | 6 |
| FCM | 13 |
| DNAm | 689340 |
| Microbiome | 15026 |
| mRNA | 1437 |
| WBC Lipidomics | 418 |
| PIs Lipidomics | 379 |
| WBC Proteomics | 1344 |
| PIs Proteomics | 1344 |

Based on DIABLO modeling, and on other assay data, the following biomarkers were identified as response indicators in a subject based on this integrative model and/or based on comparative assays (above):

### Biomarkers:

Embodiments in accordance with the present invention provide biomarkers associated with a subject's response to a vaccine. As described herein, biomarkers are the amount of RNA and/or protein, for example, of an identified target that showed an increase or decrease in subjects that later were found to have responded to a vaccine in two or less, and more typically, one dose. The increase or decrease of a biomarker can be in relation to the value for all subjects evaluated for the same biomarker, i.e., control. The biomarker can also be identified using the integrative model, as described herein.

These biomarkers are universal for all vaccines and represent targets for identifying subjects that will respond to a vaccine in a more robust fashion, as compared to non-responders. Testing was performed, in part, using differential gene expression and antibody titers after the first, second and third boosts (if necessary). Protein and RNA levels were determined using conventional assays, known to the industry. The data obtained was then used to identify biomarkers via one of the manners previously described herein.

In some aspects, combinations or networks of markers were indicative of a subject who would respond to a vaccine, or alternatively, would require additional vaccinations to obtain a more robust outcome.

In one embodiment, the biomarker is transcriptional suppressor GFI1, which is upregulated in responders, as compared to non-responders, controls or was identified using the integration model.

In another embodiment, the biomarker is transcriptional suppressor GFI2, which is upregulated in responders, as compared to non-responders, controls or was identified using the integration model.

In another embodiment, the biomarker is transcriptional suppressor GF15, which is upregulated in responders, as compared to non-responders, controls or was identified using the integration model.

In another embodiment, the biomarker is surface receptor FCGR3A (CD16a), which is downregulated in responders, as compared to non-responders, controls or was identified using the integration model.

In another embodiment, the biomarker is the Fc fragment of the IgE receptor, FCER1A, which is upregulated in responders, as compared to non-responders, controls or was identified using the integration model.

In another embodiment, the biomarker is the autoimmune regulator protein (AIRE gene), which is upregulated in responders, as compared to non-responders, controls or was identified using the integration model.

In another embodiment, the biomarker is the CD8a antigen, which is upregulated in responders, as compared to non-responders, controls or was identified using the integration model.

In another embodiment, the biomarker is the CD8b antigen, which is upregulated in responders, as compared to non-responders, controls or was identified using the integration model.

In another embodiment, the biomarker is LGALS3 (suppresses CD8 T-cells), which is downregulated in responders, as compared to non-responders, controls or was identified using the integration model.

In another embodiment, the biomarker is ribosomal genes, which is downregulated in responders, as compared to non-responders, controls or was identified using the integration model.

In another embodiment, the biomarker is CD28, which is upregulated in non-responders, as compared to responders, controls or was identified using the integration model.

In another embodiment, the biomarker is the CD3-e gene, which is upregulated in non-responders, as compared to responders, controls or was identified using the integration model.

In another embodiment, the biomarker is poly IC, which is upregulated in non-responders, as compared to responders, controls or was identified using the integration model.

### Cytokine Levels as a Biomarker:

Embodiments herein also provide a number of cytokine biomarkers for use as indicators of a responder. Biomarkers identified using embodiments herein include: IL-8, IFN-Υ, IL-2, IL-1β, IL-6, IL-5, IL-7, IL-10, IL-17A, TNF-α, IL-12 p40, GM-C SF, and IL-15.

### DNA Methylation as a Biomarker:

Embodiments in accordance with the present invention provide epigenetic biomarkers associated with responders to a vaccine. As described herein, biomarkers can be levels of methylation at identified genes that associate with vaccine responders (as compared to controls). In one aspect, CPGs were tested for hypermethylation in a number of target sites. The RPS14 ribosomal gene was identified as being hypermethylated and being a biomarker for a responder to a single dose of vaccine. In addition, and as a likely result of the RPS14 hypermethylation, the ribosomal genes were identified as being downregulated in responders. As noted above, DNA methylation biomarkers were identified using a comparison against a control, against non-responders or through the use of the integration model, described herein.

mDC, pDC and T Cells:

Embodiments in accordance with the present invention provide that certain pre-vaccine cell populations correlate with being a responder. Here it has been found that responders have an increase in the total number of mDCs (myeloid DCs) and/or pDCs (plasmacytoid DCs) as compared to control levels, and that responders have an increased number of T cells as compared to control levels. As such, the number of mDCs, pDCs and T cells are biomarkers for identifying a responder. Further, mDCs that express either CDKN1 and/or NDRG2 as associated with distinct mDC subsets related to responders (using scRNA-seq to identify). The correlation in mDC is also enhanced for a responder, where the mDCs express high levels of HLA class II. Finally, the ratio of CDKN1 to NDRG2 can be used to identify a responder to a vaccine.

### CD25+OX40+:

In another embodiment, subjects having an increased number of antigen-specific CD4 T cells, as compared to an average number for a subject in the population, are subjects who will respond to a vaccine. In one aspect, surface activation induced markers (AIM) CD25 and OX40 are utilized as markers for antigen-specific CD4 T cells. It has previously been shown that these two markers are indicative of a cell that is an antigen-specific CD4 T cell. These AIMs are specific for antigen-specific CD4 T cells as compared to other T cell surface antigens. See Reiss et al., Comparative analysis of activation induced marker (AIM) assays for sensitive identification of antigen-specific CD4 T cells, 2017, PLoS ONE 12(10): e0186998. Identification of an increase in antigen-specific CD4 T cells has been shown herein to be predictive of a subject who will respond to a vaccine.

In some aspects, peripheral blood from an unvaccinated subject is harvested, and harvested cells run through a cell sorter. In one embodiment, a cell sorter is programed to identify cells that are antigen-specific CD4 T cells using appropriate cell surface markers. In one aspect, antigen-specific CD4 T cells are CD25+OX40+. Subjects peripheral blood are then re-checked for the same surface antigens after a first vaccination using a conventional vaccine, for example, a vaccine for the influenza virus. Frequencies of the cells in each subject were followed over the course of 6 months. Subjects having a higher frequency of CD25+OX40+ cells pre-vaccination were shown to have a higher likelihood to respond to a vaccine than subjects that had average or control frequencies of the same cells. The frequency of CD25+OX40+ cells was also indicated to be a biomarker using the integration model, describer herein.

In one embodiment, a method for identifying a vaccine responder is provided. In a first operation, a subject in need of the appropriate vaccine has a sample taken and tested for the presence of antigen-specific CD4 T cells. In a second operation, the frequency of antigen-specific CD4 T cells is compared to a median frequency from the same demographic of subjects. In a third operation, subjects that have an increase in the frequency of antigen-specific CD4 T cells are vaccinated a first time with a convention dose for the vaccine. In a fourth operation, subjects having a frequency of antigen-specific CD4 T cells that is equal to or below the control level, are vaccinated at least one more time, at an appropriate length of time from the first vaccination. In one aspect of the method, an antigen-specific CD4 T cell is a cell having CD25+OX40+.

### LN FNA Biomarkers:

In some embodiments, increased levels of PD-1+ CXCR5+ cells and CD20+CD38+ B cells in the axillary and inguinal LNs are indicative of a responder. In other embodiments, increased levels of CD38+CD20+ cells in axillary lymph nodes are indicative of a responder. These biomarker cells show a higher level in these samples than corresponding controls, or were identified using the integrative computational modeling described herein.

### Metabolites:

In some embodiments, metabolites may act as biomarkers for identification of a responder. In one aspect, the metabolite is triacylglycerol, in another sphingomyelin 44, in still another, hexosylceramide, and in still another, ceramide. Each of the metabolites is elevated in a responder as compared to a control or was identified using the integrative computational modeling described herein.

### Integrative Network of Biomarkers

In some embodiments, identifying a subject that will respond to a first dose of vaccine requires analysis and integration of a network of biomarkers. In addition, a network of biomarkers can be used as the baseline starting point for a subject prior to vaccination, such that the subject is administered one or more immune modulators to establish a baseline starting point in line with the identified network of biomarkers. The network of biomarkers provides a signature for improved outcome with relation to a subject's immunity and how that subject will respond to a vaccine.
In one aspect, a pair of samples from a subject, one taken 14 days prior to vaccination, and the other taken 14 days post-vaccination, can undergo a series of testing, including a combination of epigenetic studies, RNA sequencing, Milieu Interieur, Proteomics, WBC Metabolomics and proteomics, flow cytometry, and microbiome testing. Each of the tests is used to identify potential biomarkers that could be associated with an immune response, and differences or trends between the pre- and post-vaccination analysis identified. The total number of identified biomarkers is the network of biomarkers. At the same time, the sample taken 14 days post vaccination is also tested for an appropriate antibody titer to identify whether the subject responded to the vaccination or did not respond to the vaccination. A titer of at least 3 to 10 mIU/ml is indicative of an acceptable antibody titer for a responder, and a titer of greater than 10 mIU/ml is considered positive for identification of a responder.
The network of indicators includes two or more, three or more, four or more, five or more, six or more, and the like, of the previously described biomarkers herein. Biomarkers were identified, in some embodiments, by obtaining the data, and using a integrative model that maximizes the covariance between all the data at the pre-immunization and log(antibody titer) at post-immunization.
The network of biomarker indicators, as identified using the combination of testing procedures above, can then be cataloged to correlate to a responder or non-responder based on the antibody titer (below 3 mIU/ml for a non-responder), and the testing repeated on additional subjects.
In one aspect, the network of biomarkers includes a combination of a ratio of DC2 to DC4 cells, and ratio of NDRG2 to CDKN1 genes.

### Methods:

Embodiments herein include methods for identifying a subject that will respond to two or fewer doses of vaccine, and more typically, a single dose of vaccine. In one aspect, the method comprises: isolating a sample from a subject in need of a vaccine; contacting the sample with reagents specific for each of the biomarkers described herein to assess the appropriate biomarker parameter, e.g., expression level, methylation status, cell numbers, etc.; determining whether the sample biomarker has an increased or decreased expression level as compared to the same biomarker in a control sample, or alternatively, running the biomarker through the integration model to maximize covariance of the data; and determining whether the sample biomarker is indicative that the subject is a vaccine responder. In some cases, the method includes contacting the sample with multiple reagents specific for two or more biomarkers, three or more biomarkers, four or more biomarkers, and the like, to provide a network of biomarkers useful in identifying a subject that will respond to a vaccine.

In some aspects, the sample is peripheral blood. In other aspects, the sample is mDCs. In yet other aspect, the sample is lymph or lymph node aspirate. In other aspects herein, the biomarker is the transcriptional suppressor GFI1, and the GFI1 is increased as compared to a control level of GFI1. In another aspect herein, the biomarker is the transcriptional suppressor GFI2, and the GFI2 is increased as compared to a control level of GFI2; in yet another aspect herein, the biomarker is the transcriptional suppressor GF15, and the GF15 is increased as compared to a control level of GF15. In some cases, the biomarker is the surface antigen CD8a, and the CD8a is increased as compared to a control level of CD8a. In other cases, the biomarker is the surface antigen CD8b, and the CD8b is increased as compared to a control level of CD8b. In still other cases, the biomarker is LGALS3, and the LGALS3 is decreased as compared to control levels of LGALS3. In still other aspects, the biomarker are ribosomal genes, and the ribosomal genes are down regulated as compared to ribosomal genes in control samples. It is also envisioned that the biomarker can relate more directly to a non-responder subject, for example, through the use of differential gene expression. In one aspect, the differential gene expression is for the CD28 gene and for the CD3-e gene.

In another aspect, embodiments herein include methods for identifying a vaccine responder. The method comprises: isolating a sample from a subject in need of a vaccine; running the sample through a flow cytometer to identify CD25+OX40+ T cells; and determining whether the number of CD25+OX40+ cells in the sample is a positive or negative indicator of a vaccine responder.

Embodiments herein also include methods for preparing biomarker assays for identifying a vaccine responder. In one aspect, the method comprises: taking a pre-vaccination sample from a known group of subjects; taking a post-vaccination sample from the same group of subjects, the pre- and post-vaccination samples being from the same source; evaluating each pre- and post-sample for differential gene, protein, and RNA expression to provide a battery of potential biomarker targets; identifying biomarkers that showed differential expression, and correlating to subjects that responded to the vaccine by antibody titer to the vaccine; and determining biomarkers that are indicative of a responder.

Embodiments herein also include methods for altering a subject's biomarker levels to alter or improve the outcome of a vaccination. In this embodiment, one or more samples are recovered from a subject in need of a particular vaccination; the samples are recovered prior to the vaccination, and a series of two or more, three or more, four or more, and the like, assays are performed on the samples to identify a baseline set of biomarkers, as described herein the subject's baseline network of indicators are then compared to the same indicators for a vaccine responder, and a determination on whether the subject requires immune modulators to conform his or her biomarker network to a responders network; where required, the subject is administered one or more immune modulators to conform the subject's biomarker network to a responder's biomarker network. In one embodiment, the immune modulator is poly IC, a TLR agonist. In some aspects a second sample is taken from the subject after a predetermined amount of time to track the modifications of the subject's network of biomarkers, and where appropriate, challenged with a second administration of immune modulators. The vaccine is administered once the subject's biomarker network is comparable to a responder's biomarker network.

Methods herein also include vaccine design wherein a vaccine is designed through alternations in the antigen and/or adjuvant of the vaccine to elicit a setpoint network of biomarkers associated with vaccine response. Using the integration model, one or more of the biomarkers described herein can be used to track the effectiveness of a vaccine. Alterations in the type or amount of antigen, composition and amounts of the adjuvant, timing of the administration can all be used to maximize the effect on the setpoint biomarkers associated with a responder.

### Kits:

Embodiments herein also include a kit for assessing the expression, number or amount of a biomarker listed herein, which can then be used to identify whether a subject is a responder to vaccines. The kit herein comprises a plurality of reagents, each required to ascertain the expression, number or amount of a biomarker. For example, reagents can include antibodies (or antibody derivatives, fragments, chimerics), and the like, against CD8a for example or oligonucleotide primers necessary for PCR, probes, and other necessary reagents. Reagents may be provided in any useful form, including bound or fixed to solid state substrates. The kits may also include components useful for performing the methods herein, including fluids, containers, instructional materials, and the like.

The following examples are intended to further illustrate the invention and its preferred embodiments. They are not intended to limit the invention in any manner Examples:

### Example 1: Predicting Final Antibody Titer from Pre-Immunization Data

A series of pre-vaccination and post-HBV vaccination samples were taken from 15 subjects. A number of assays were performed on each sample including, flow cytometry, DNA methylation, mRNA, microbiome, WBC lipodomics and PLS lipodomics. An integration model was used to identify key molecular drivers from across the above described data set. The integrative model, Latent cOmponents (Singh et al., DIABLO: an integrative approach for identifying key molecular drivers from multi-omics assays, Bioinformatics, 2019, 1-8, incorporated in its entirety for all purposes), identified the most predictive features of final Ab titers for the pre-vaccination set of samples, and then applied these features to predict the actual titers from the same subjects in the post-HBV vaccinated samples.

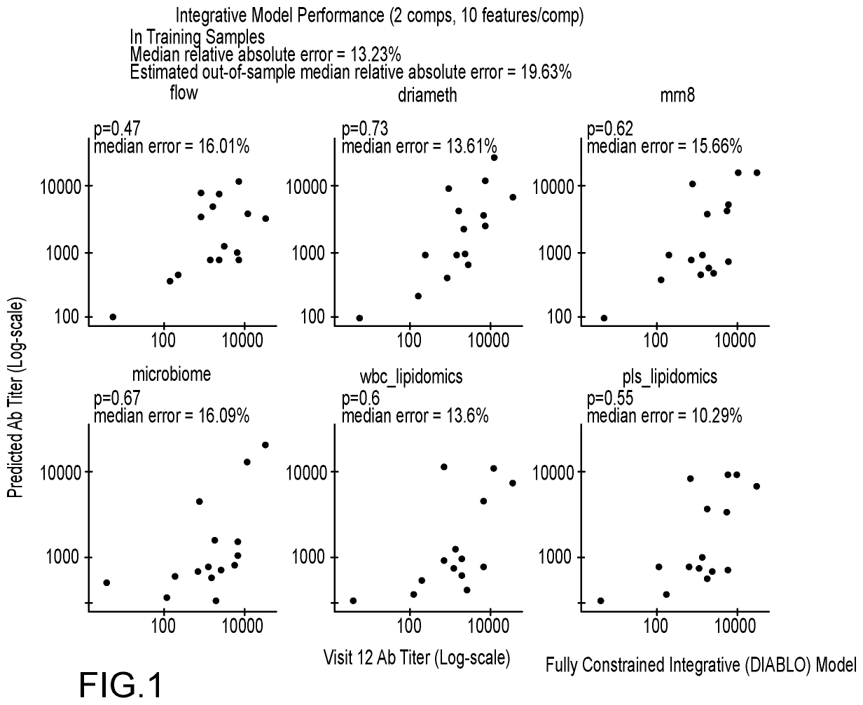

As shown in FIG. 1, the predicted versus actual antibody titers can be visualized in a series of regression plots, where the dotted line depicts perfect prediction accuracy. The overall median relative error rate of the integrative model was about 13%. The median relative error rate corrected for the model's optimism was about 20% (estimated by cross-validation). The biomarkers identified were enriched for genes involved regulation of cytokine production and maturation of B-cells to plasma cells.

The data in FIG. 1, shows that modeling can be performed that predicts the final antibody titers from pre-immunization data.

### Example 2: qPCR Summary for Responder Identification

10 subjects were challenged with the medically approved HepB vaccine. Blood samples, 50 or 100 ml, were collected at D0, D1, D3, D7 and D14. Cells were sorted into monocytes, mDC, pDC, Natural Killer cells and Neutrophils. Serum Ab titers were measured for each subject after the first vaccine dose. Based on serum Ab titers, there were 3 responders to the HepB vaccine and 7 non-responders to the HepB vaccine.

As described above, the Data Integration Analysis for Biomarker Discovery Using Latent cOmponents model was used to maximize the covariance between all the data at the pre-immunization visit and log(antibody titers) at the final visit using CDKN1 and NDRG2-associated CpGs and transcripts only.

NDRG2 and CDKN1 were assessed by qPCR in the responders and non-responders, such that 4,460 sorted mDCs were tested. Approximately 1,779 single mDC cells expressed at least one of the two markers. In addition, DNA methylation was determined for each of the two markers in the cells expressing at least one of the markers. The integrative model was used to maximize the covariance between the data related to DNA methylation and transcript expression.

CDKN1 and NDRG2 were found to be associated with distinct mDC subsets related to responder status in scRNA-seq. Their ratio in whole blood RNA-seq is significantly associated log(antibody titers) at the final visit. Querying MSigDB was also used to determine that the two biomarkers, NDRG2 and CDKN1, are differentially regulated in mDC stimulated in vitro with TLR3 agonist poly I:C. The median relative error rate for the tests was about 21%.

As a result, single cell RNA sequencing revealed cellular complexity and revealed that there were multiple mDC and pDC subsets of cells. Using qPCR of selected subset-specific biomarker genes, the inventors found that the ratio of the two mDC subsets correlated with response to a single dose of the HepB vaccine. A relative high level of the mDC subset that expresses high levels of HLA class II appears to predict vaccine response. Pre-conditioning a subject to increase the relative proportion of this mDC subset should increase vaccine efficacy.

### Example 3: Baseline Predicts Outcome for Hepatitis B Vaccination

Approximately 30% of the world's population (i.e., about 2 billion persons) have serologic evidence of Hepatitis B virus (HBV) infection. Schillie et al., Seroprotection after recombinant hepatitis B vaccination amount newborn infants: a review. Vaccine. 31, 2506-2516. As the leading cause of chronic hepatitis and cirrhosis, HBV causes significant morbidity and mortality worldwide. Each year, approximately 620,000 HBV-infected persons die from chronic liver disease P. Van Damme, In Pediatric Vaccines and Vaccinations, A European Textbook, T Vesikari and Damme Eds., Epub (2017), pp 109-116. The WHO recommends universal neonatal/infant HBV immunization, as well as vaccination of adults at risk for HBV WHO, Hepatitis V vaccines. Wkly Epidmiol Rec 84, 405-419 (2009). All current HBV vaccines are composed of hepatitis B surface antigen (HBs) and employ a 2- to 4-dose vaccination schedule Shepard et al., Hepatitis B virus infection: epidemiology and vaccination, Epidemiologic reviews 28, 112-125 (2006). The serologic correlate of protection (CoP) against chronic HBV infection is one of the best-defined CoP for any vaccine as an antibody level of ≥10 mIU/mL anti-HBs antibody (defined by the WHO Anti-HBs Reference Preparation) Nakaya et al., Systems biology of vaccination for seasonal influenza in humans. Nat Immunol 12, 786-795 (2011). Importantly, there is a direct correlation between quantitative antibody level and protection Schillie et al., Seroprotection after recombinant hepatitis B vaccination amount newborn infants: a review. Vaccine. 31, 2506-2516. Some achieve an anti-HBs response of ≥10 mIU/mL after one or two doses of vaccine already; specifically, 30-50% after 1 dose and 50-75% after two doses Mast et al., Hepatitis B vaccine, J Plotkin, H Orenstein Eds., Vaccines (Saunders, Philadelphia, ed. 4^{th}, 2004). Unfortunately, persistent non-responders, 5-10% of the vaccinated population, stay unprotected, even after a completed vaccination schedule; the reason for this failure is complex Leuridan et al., Hepatitis B and the need for a booster dose, Clin Infect Dis 53, 68-75 (2011).

We advanced this concept applying one of the most comprehensive study ever conducted in the systems vaccinology arena (whole blood transcriptomics and epigenetics, plasma metabolomics, lipidomics and proteomics, as well immune cell phenotyping and microbiome analysis) and using a novel integrative analysis platform for multi-omics (DIABLO). In this study conducted in HBV-seronegative healthy adults aged 40-80 years, we found that the key genes involved in the vaccine response were differentially expressed at baseline between HBV vaccine responders versus those who did not respond after either one or 3 doses of the vaccine. Baseline differences in the particular pathways that separated the two response groups were consistent with epigenetics profiling, as well as in plasma proteomic analysis and peripheral blood immune cell subset frequencies in circulation at the time of vaccination Integrating these various omic data sets, predictive models could be built that predict the anti-HBs response following 3 doses of HBV vaccination using only pre-vaccine baseline data. Furthermore, preliminary evidence from our recent study suggests that predictive signatures for subjects generating protective immunity after a single immunization could also already be defined at baseline.

### Example 4: Baseline Predicts Outcome for Malaria Vaccination

Malaria remains a major global health problem, causing an estimated 219 million cases and 435,000 deaths in 2017 alone W.M.R. 2018. (WHO, Geneva, 2018), vol. 2019. Decades of research have thus far only resulted in a single subunit vaccine candidate completing a phase 3 trial for licensure, the RTS,S/AS01E or Mosquirix™. Despite being recommended by the WHO for pilot implementation studies starting in 2019 in Africa, the efficacy of the vaccine against clinical malaria is moderate and of limited duration S.C.T.P. RTS, Efficacy and safety of the RTS/AS01 malaria vaccine during 18 months after vaccination: a phase 3 randomized, controlled trial in children and young infants at 11 African sites. PloS Med 11, e1001685 (2014). At present it is not known why the RTS,S vaccine only protects a proportion of immunized subjects, specifically 30-55% over one year and 26-36% with a booster dose over 3-4 years. Vaccines based on attenuated Plasmodium falciparum: parasites through irradiation (Ishizuka et al., Protection against malaria at 1 year and immune correlates following PfSPZ vaccination. Nat Med 22, 614-623 (2016)) or chemoprophylaxis (Roestenberg et al., Protection against a malaria challenge by sporozoite inoculation., N Engl J Med 361, 468-477 (2009)) are promising alternatives that have shown up to 100% efficacy in clinical trials in naïve adults; however, efficacy under natural malaria exposure in the field appears to be lower. Jongo et al., Immunogenicity, and protective efficacy against controlled human malaria infection of Plasmodium falciparum Sporozoite vaccine in Tanzanian adults Am J Trop Med Hyg 99, 338-349 (2018). Major knowledge gaps in the development of more effective malaria vaccines are the absence of immune correlates of protection and understanding of the mechanisms for protective immunity. The best correlates for either of these malaria vaccines is the titer of IgG antibodies against the main vaccine antigen (the circumsporozite protein, CSP), which are thought to prevent liver stage infection and thus subsequent blood stage infection causing clinical disease. However, unlike for anti-HBs following HBV, the positive predictive value of vaccine-induced IgG titers is too low to be considered as CoP Ubillos et al., Baseline exposure, antibody subclass, and hepatitis B response differentially affect malaria protective immunity following RTS,S/AS01E vaccination in African children. BMC Med 16, 197 (2018).

Of relevance to the concept of the immune baseline at time of vaccination predicting vaccine outcome, in large RTS,S phase 2b trials in African children, peripheral blood monocyte-to-lymphocyte ratios at baseline negatively correlated with RTS,S/AS01 efficacy Warimwe et al., Peripheral blood monocyte-to-lymphocyte ratio at study enrollment predicts efficacy of the RTS,S malaria vaccine: analysis of pooled phase II clinical trial data, BMC Med. 11:184., 10.1186/1741-7015. Applying systems biology to peripheral blood samples from individuals vaccinated with RTS,S/AS01E (African children) (S.C.T.R. Partnership, S.C.T.P., efficacy and safety of RTS,S/AS01 malaria vaccine with or without a booster dose in infants and children in Africa: final results of a phase 3, individually randomized controlled trial, Lancet 386, 31-435 (2015)) or whole sporozoites under chemoprophylaxis in malaria-naïve adults (Bijket et al., Cytotoxic markers associate with protection against malaria in human volunteers immunized with Plasmodium falciparum sporozoites, J Infect Dis 2010, 1605-1615 (2014)) we aimed to identify immune signatures that could predict vaccine immunogenicity and protection in order to decipher mechanisms relevant for vaccine responsiveness. We analyzed the transcriptomic profile of blood samples after in vitro recall responses with the CSP and P. falciparum-infected erythrocytes at baseline and at 4 months and 1 month post-immunization with chemoattenuated parasites and RTS,S/AS01E vaccines, respectively. Several signatures of protection against malaria for both immunization strategies were identified post-vaccination but importantly, also at baseline with high generalization capabilities (up to 87%) and accuracies (up to 100%). The resulting biomarker signatures were then validated with an independent chemoattenuated sporozoites trial and a separate set of RTS,S vaccinated children and final predictive models deduced using artificial neural networks predicted protection with accuracies up to 100%. Although most genes contained in predictive baseline signatures differed between immunization modes, common elements centered around innate immune status where immune activation and inflammation appeared centrally relevant (as evidenced by the canonical and non-canonical NFkB pathway, among others).

### Example 5: Baseline Predicts Outcome for Other Vaccines and Beyond

The recognition that ‘the first flu is forever’, i.e. the first early-life exposure to influenza virus will partly determine the response to and protection against different influenza strains for the rest of a person's life (Viboud et al., First flu is forever. Science 354, 706-707 (2016)), suggested that baseline can determine outcome for infection as well as vaccination. The concept that “baseline predicts outcome” also lends itself to precision public health, i.e. at the population level as is already done with influenza vaccines every year, where different vaccines and/or different numbers of doses are administered depending on age of the individual and prior vaccine status (Grohskopf et al., Prevention and Control of Seasonal Influenza with Vaccines: Recommendations of the advisory committee on immunization practices—United States, 2018-2019MMWR Recomm Rep 67, 1-20 (2018). For example, a 3-year old child receives 1 (if previously vaccinated) or 2 (if vaccine naïve) doses of either a quadrivalent intranasal live attenuated vaccine or an inactivated vaccine, whereas an individual aged >65 years may receive a single dose of standard trivalent inactivated vaccine, high-dose vaccine or a formulation which includes the MF59 adjuvant.

That baseline can predict outcome also holds true beyond vaccine responses. For example, a human cohort study applying a multi-omic approach identified that the in vitro response to stimulation with 20 different pathogens can be accurately predicted from baseline omic parameters Bakker et al., Integration on multi-omics data and deep phenotyping enables prediction of cytokine responses. Nat Immunol 19, 776-786 (2018). A similar predictive paradigm forms a large part of the raison d'être of the Mileu Interior project, which has shown that in vitro immune responses to a range of infectious stimuli can be predicted from baseline data Scepanovic et al., Human genetic variants and age are the strongest predictors of humoral immune responses to common pathogens and vaccines. Genome medicine 10, 59 (2018). Whether baseline immune status can predict clinical outcome for infectious diseases remains largely unexplored. The only direct evaluation of such association that we are aware of stems from one of our recent studies demonstrating that the risk for severe infections early in life can be predicted from baseline innate immune phenotypes measured at birth. Indirect evidence suggests the baseline concept may have much farther reach. For example, Ebola has a case fatality rate of approximately 50%, with some succumbing to disease yet others recovering, or even controlling the infection in an asymptomatic manner Malvy et al., Ebola virus disease. Lancet 393, 936-948 (2019). Is the differential pathogenesis of Ebola infection also due to baseline immune-omic signatures, and if so, could immune modulators shift the curve from low responders (those who will die from Ebola) to high responders (those who will remain asymptomatic)? Similarly, HIV infection has a bell-shaped pathogenesis curve ranging from rapid progressors, to moderate progressors, to elite controllers Goulder et al., HIV control, is getting there the same as staying there? PLoS pathogens 14, e10072222 (2018). Could predictive signatures for HIV immune responses be identified, leading to novel prevention and control measures? Furthermore, this concept is not limited to infectious diseases. While there is an ongoing revolution in cancer immunotherapy due to checkpoint inhibitors, less than 15% of subjects across the spectrum of cancers are currently gaining benefit from checkpoint immunotherapy, suggesting that predictive signatures could identify responders from non-responders before commencing therapy.

### Example 6: Modulating Baseline Modulates Vaccine Outcome

The extension of the baseline concept, namely that ‘modulating baseline can modulate vaccine outcome’ has enormous translational implications. Indirect evidence of this representing a feasible avenue to consider in the quest to improve vaccine outcome exists. For example, Cytomegalovirus (CMV) infection is known to activate multiple components of the innate and adaptive immune systems, and is associated with enhanced immune response of young, healthy humans to influenza vaccination; this, however, was not observed in older adults, suggesting that the impact of CMV may be age-specific. Furman et al., Cytomegalovirus infection enhances the immune response to influenza. Science translational medicine, 7 281ra243 (2015. In contrast, Epstein-Barr virus (EBV) infection, also known known to activate multiple components of the innate and adaptive immune systems is associated with reduced responses to infant vaccines. Indeed, it is not clear how basal immune activation modulate immune responses to subsequent vaccination. For example, an activated immune state prior to vaccination can reduce the response to the Yellow Fever vaccine, which is consistent with one of the studies on hepatitis B vaccination cited above where a higher inflammatory state at baseline predicted a poorer response to vaccination. Leuridan et al., Hepatitis B and the need for a booster dose. Clin Infect Dis 53, 68-75 (2011). Furthermore, administration of the immune suppressant rapamycin has been shown to increase the immune response to influenza vaccination in older adults. These observations suggest that modulating baseline can differently modulate vaccine outcome, depending on the vaccine and on the population. Alter et al., Beyond adjuvants: Antagonizing inflammation to enhance vaccine immunity. Vaccine 33 Suppl 2, B55-59 (2015).

Lastly, even vaccination can alter immune baseline and, with that, outcome for other vaccines. For example, the BCG vaccine is one of the most widely used vaccines worldwide. In addition to providing moderate protection against TB, it can have non-specific (heterologous) immunomodulatory effects beyond the target (TB), including on other vaccines. Specifically, BCG can increase the response to HBV, polio type 1, pneumococcus, and influenza vaccination. Ota et al., Influence of Mycobacterium bovis bacillus Calmette-Guerin on antibody and cytokine responses to human neonatal vaccination. J Immunol 168, 919-925 (2002). The underlying mechanisms for BCG's impact on other vaccines have not been fully elucidated but could involve reducing the baseline immune status. Freyne et al., Neonatal BCG vaccination influences cytokine responses to toll-like receptor ligands and heterologous antigens. J Infect Dis 217, 1798-1808 (2018). Vaccination as a means of modulating immune baseline has even been suggested to occur before birth, as MF59-adjuvanted influenza vaccination during pregnancy was found to alter the immune cytokine production profile in the nasal mucosa of 4-week-old infants Bischoff et al., Airway mucosal immune-suppression in neonates of mothers receiving A(H1N1) pnd09 vaccination during pregnancy. Prediatr Infect Dis J 34, 84-90 doi: 10 1097/INF.000000000000529 (2015). While the implications of this for infant vaccine responses have not been investigated, these data highlight that this concept is highly relevant for maternal immunization as well. Marchant et al., Maternal Immunisation: collaborating with mother nature. Lancet Infect Dis 17 e197-e208 (2017). In summary, infection and even other vaccines may impact the response to subsequent vaccination via modulation of the baseline immune status.

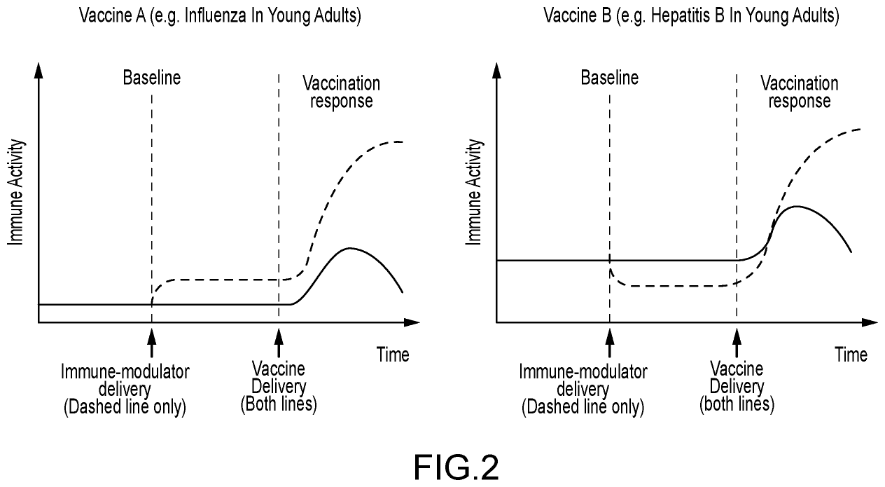

As shown in FIG. 2, for Vaccine A, which could be influenza vaccination in the young adult, an immune modulator that increases immune baseline (blue dashed line) could increase final vaccine-induced immune response. For Vaccine B, which could be hepatitis B vaccination, an immune modulator that decreases immune baseline (blue dashed line) could increase final vaccine-induced immune response.

What is claimed is:

1. A method for immunizing a subject in need thereof, comprising:
modulating two or more of the subject's immune system biomarkers to a predetermined response level to facilitate a response to a vaccine; and
administering the vaccine to the subject in a dose appropriate matter;
wherein the predetermined response level is based on parameters associated with an average vaccine responder.

2. The method of claim 1, further comprising:
testing a value for the two or more of the subject's immune system biomarkers prior to modulating the same two or more of the subject's immune system biomarkers.

3. The method of claim 2, wherein:
the two or more of the subject's immune system biomarkers are selected from the group consisting of: GFI1, GFI2, LGALS3, CD8A, CD8B, GR15, NDRG2, RPS14, CDKN1, FCGR3A, FCER1A, IL-8, IFN-2.

4. The method of claim 2, wherein:
the two or more of the subject's immune system biomarkers comprise an amount of CD25+OX40+ T-cells in the subject.

5. The method of claim 2, wherein:
the two or more of the subject's immune system biomarkers comprise at least the level of methylation at the RPS14 ribosomal gene.

6. A method for preventing a disease state in a subject comprising:
administering one or more immune modulators to the subject to positively influence a network of biomarkers;
allowing a predetermined amount of time to transpire to allow the positive influence to occur to the network of biomarker; and
administering a vaccine that prevents the disease state in the subject to the subject.

7. The method of claim 6, wherein the disease state is hepatitis or influenza.

8. A method of risk prediction of a subject's ability to mount an immune response to a singular vaccination event, the method comprising:
testing the subject for the presence and/or amount of two or more response mediated effectors;
comparing the values of the two or more response mediated effectors in the subject against a control value for the same two or more response mediated effectors; and
assign a risk prediction for mounting an immune response by the subject based on the comparison;
wherein, the subject can be assigned to a category of vaccine responder or vaccine non-responder.

9. The method of claim 8, wherein:
the two or more response mediated effectors is selected from the group consisting of IL-8, IFN-Υ, IL-2, IL-1β, IL-6, IL-5, IL-7, IL-10. IL-17A, IL-12, TNF-α, p40, GM-C SF, IL-15, CD28, LGALS3, CD8A, CD8B, GFI1, GFI2, GFI5, methylation state of RPS14, pDC expression of CDKN1 and NDRG2, number of CD4+OX40+ T Cells, number of PD-1+ CXCR5+ B Cells and CD20+CD38+ B Cells, triacylglycerol, sphingomyelin 44 and hexosylceramide.

10. The method of claim 9, wherein the testing the subject involves testing for the presence and/or amount of three or more response mediated effectors.

11. A method of identifying whether a subject would respond to a vaccination: (a) obtaining a sample from the subject prior to the vaccination; (b) measuring a protein or transcriptional expression level for GFI1 in the sample; and (c) generating a comparison of the protein or transcript level of GFI1 in the sample to a reference or control level of the GFI1, and using the comparison to predict whether the subject would respond to the vaccination, wherein an increase in GFI1 levels in the sample as compared to the reference sample shows that the subject would respond to one vaccination.

12. The method of claim 11, further comprising: measuring a protein expression level for CD8a and for CD8b in the sample and comparing the protein level in the sample to a reference level of CD8a and CD8b, wherein an increase in CD8a and CD8b in the sample as compared to the reference sample shows that the subject would respond to one vaccination.

13. The method of claim 11, further comprising: measuring a protein expression level for LGALS3 in the sample, and comparing the protein level in the sample to a reference level of LGALS3, wherein a decrease in LGALS3 in the sample as compared to the reference sample shows that the subject would respond to one vaccination.

14. The method of claim 11, further comprising: measuring a protein expression level for GFI2 in the sample, and comparing the protein level in the sample to a reference level of GFI2, wherein an increase in GFI2 in the sample as compared to the reference sample shows that the subject would respond to one vaccination.

15. A method for treating influenza in a patient: (a) obtaining a biological sample from the patient; (b) testing the biological sample for multiple biomarkers using an assay that detects at least one biomarker selected from the group consisting of GFI1, GFI2, CD8a and CD8b, and detecting the presence, absence or quantity of such biomarker; (c) based on the presence of an increase in the at least one biomarker, as compared to a corresponding reference biomarker, administering a vaccine to treat the influenza.

16. A multiplexed assay kit configured to identify a responder to an influenza vaccine, the kit comprising: (a) a solid state assay for detecting the levels of one or more of GFI1, GFI2, GRI5, LGALS3, FCGR3A, FCER1A, CD8a, CD8b, NDRG2, RSP14, and CDKN1; and (b) a reference standard for a non-responder level for each of the one or more of GFI1, GFI2, GRI5, LGALS3, FCGR3A, FCER1A, CD8a, CD8b, NDRG2, and CDKN1; wherein the kit is configured to determine the level of GFI1, GFI2, GRI5, LGALS3, FCGR3A, FCER1A, CD8a, CD8b, NDRG2, RPS14, and CDKN1 and that the levels as compared to the reference standard identify a responder.

17. The kit of claim 16, wherein the responder requires only one dose of the influenza vaccine.

18. The kit of claim 16, wherein the non-responder requires multiple doses of the influenza vaccine.

19. The kit of claim 16, wherein the detecting the levels further comprises identifying the level of methylation for one or more ribosomal genes.

20. The kit of claim 19, wherein the ribosomal gene is RPS14.
